(19) United States
(12) Patent Application Publication
(10) Pub. No.: US 2023/0414306 A1
(43) Pub. Date: Dec. 28, 2023

(54) INTEGRATED ROBOTIC INTRAOCULAR SNAKE

(71) Applicant: The Johns Hopkins University, Baltimore, MD (US)

(72) Inventors: Iulian I. Iordachita, Lutherville-Timonium, MD (US); Makoto Jinno, Baltimore, MD (US)

(21) Appl. No.: 18/037,508

(22) PCT Filed: Nov. 17, 2021
(86) PCT No.: PCT/US21/59606

§ 371 Date: May 17, 2023

Related U.S. Application Data
(60) Provisional application No. 63/114,984, filed on Nov. 17, 2020.

(51) Int. Cl. A61B 34/00; A61F 9/007

(52) U.S. Cl. CPC A61B 34/71; A61F 9/00727; A61B 2034/301

(57) ABSTRACT

A dexterous manipulation device is provided that includes disc element and actuation wires. In preferred systems, the disc elements each have a curved top surface and a corresponding curved bottom surface. The actuation wires are threaded through apertures of each disc element. In certain aspects, the disc elements are stacked alternating with the curved top and bottom surfaces of adjacent disc elements forming a rolling joint. In preferred systems, the device has a total of 45 degrees of bending motion with two degrees of freedom.

The present application claims priority of U.S. provisional application No. 63/114,984, filed Nov. 17, 2020, which is incorporated by reference herein in its entirety.

### STATEMENT REGARDING FEDERAL FUNDING

This invention was made with government support under grant 1R01EB023943 awarded by the National Institutes of Health. The government has certain rights in the invention.

### FIELD OF THE DISCLOSURE

The present disclosure relates generally to manipulator devices, and more particularly, to an integrated robotic intraocular snake.

### BACKGROUND

Retinal microsurgery refers to a family of microsurgical procedures that treat retinal diseases such as retinal detachment macular hole, diabetic retinopathy, epiretinal membrane, and with emerging application to retinal vein occlusion and others. Retinal microsurgery is one of the most technically challenging and high consequence surgical disciplines. In the operating room, a surgical microscope is positioned above the patient's eye to provide magnified visualization of the posterior of the eye, as shown in FIG. 1. Small instruments, e.g., 23 Ga with 0.65 mm diameter, are inserted through trocars on the sclera to operate at the back of the eye. The surgeon needs to control the instrument motion in a very fine and precise manner to handle the delicate eye tissue.

Due to the trocar constraint at the sclerotomy, the instrument motion is coupled with the eye movement. If the surgeon intends to keep the patient's eye still, only three rotational degrees of freedom (DOF) about the sclerotomy and one translational DOF along the instrument axis are allowed. This concept is termed as remote center-of-motion (RCM) in robotics. The lateral translation of the tool will move the patient's eye, causing change of the view in the microscope and possibly relative motion between the instrument and the retina, which is potentially risky when the instrument tip is close to the retina. This constraint limits not only the instrument workspace inside the patient's eye, but also the orientation of the instrument at a given position. A similar problem exists in laparoscopic surgery.

In retinal microsurgery, instrument dexterity at the distal end can potentially be very useful. A prototypical retinal procedure is epiretinal membrane (ERM) peeling. ERM is a thin, semitransparent layer of scar tissue that forms on the surface of the retina. It induces surface stress on the retina that results in wrinkles and striae that distort both the retinal surface and the patient's vision. In ERM peeling, the surgeon typically uses a micro-forceps tool to carefully grasp the edge of the membrane, and slowly delaminate it off of the retinal surface, as shown in FIG. 1. Besides straight instruments, angled instruments are available to enable approaching the membrane with different tool orientations, e.g., 45 degree delamination spatula and pic, as well as vertical 90 degree scissors. Incorporating additional DOFs at the distal end of the instrument, can provide more flexibility for the surgeon to grasp the membrane at the optimum angle, and to control the peeling trajectory thereby minimizing shear stress on the retina.

Another extremely difficult procedure is retinal vein cannulation (RVC) that has the potential to treat retinal vein occlusion. In this procedure a therapeutic agent, e.g., plasminogen activator (t-PA), is directly injected into the occluded vein using a micropipette. Retinal veins are typically less than 100 μm in diameter. The micropipette needs to puncture the retinal vein, and to stay within the vessel for drug delivery. FIG. 2 illustrates a simulated RVC, in which a 70 μm micropipette is used to inject air into the vessel of a chorioallantoic membrane. Using an angled micropipette, e.g., 30 degrees or aiming a straight micropipette at the vessel with an advantageous angle, e.g., 45 degrees that can allow a more gradual approach to the retina vein, and potentially improve safety by reducing the likelihood of puncturing through the retina vein. Surgical instruments such as these, with angled tips provide a suboptimal solution that requires multiple instruments, cumbersome surgical workflow, and less than optimum success rates and safety.

Certain robotic systems for retinal microsurgery have been developed to enhance natural human capabilities. The main approaches are hands-on cooperatively controlled systems, master-slave teleoperated systems, handheld robotic devices, and untethered micro-robots. The untethered micro-robots have the least constraints on workspace and manipulability, can overcome many current limitations if they can deliver sufficient force and the surgical workflow can be adapted accordingly. A pre-curved concentric nitinol tubes approach has been investigated to provide intraocular dexterity. Microstent delivery into the retinal vessel was attempted. The maximum curvature to pre-bend a nitinol tube poses the challenge on balancing the length of the dexterous wrist mechanism and the range of motion, i.e., maximum rotation angle.

### SUMMARY

The present disclosure provides an integrated robotic intraocular dexterous manipulation device that is compact in size with a detachable drive mechanism.

According to one aspect of the present disclosure, a dexterous manipulation device may include a plurality of disc elements each having a curved top surface and a corresponding curved bottom surface. In addition, the device ma include actuation wires threaded through apertures of each disc element. The disc elements are stacked alternating with the curved top and bottom surface of adjacent disc elements forming a rolling join. The device also has a total of 45 degrees of bending motion with two degrees of freedom.

In an exemplary embodiment, each disc element is about 0.2 mm thick. The apertures formed through each disc are arranged to provide a minimum contact length of about 0.7 mm between neighboring disc elements. The device may be robotically controller. Additionally, the device may be less than about 0.9 mm in diameter and the length of the stacked disc elements may be about 3 mm or less. A distal end of the device may include one of a needle tip, forceps, a pipette, an intra-ocular device, or a gripper.

According to another aspect of the present disclosure, a dexterous manipulation device may include a plurality of disc elements each having a partially curved top surface and a partially curved bottom surface corresponding to the curved top surface. Additionally, the device may include actuation wires threaded through apertures of each disc element. The disc elements are stacked alternating with the curved top and bottom surfaces of adjacent disc elements forming a rolling join. The device has a total of 45 degrees of bending motion with two degrees of freedom. In this configuration, neighboring disc elements maintain constant contact with each other. The apertures formed through each disc element are arranged to provide a minimum contact length of about 0.7 mm between neighboring disc elements. The length of the stacked disc elements is about 2 mm or less.

According to yet another aspect of the present disclosure, a surgical system is provided. The system may include a dexterous manipulation device that includes at least one bending portion actuated by wires and a drive mechanism mounted at a right angle relative to an actuation direction of the dexterous manipulation device. The bending portion may include a plurality of disc elements each having a curved top surface and a corresponding curved bottom surface and the wires may threaded through apertures of each disc element.

In an exemplary embodiment, the drive mechanism is detachable from the dexterous manipulation device. The system may further include a body unit mated between the drive mechanism and the dexterous manipulation device. The drive mechanism may further include a housing, a motor within the housing, and a plurality of pulleys.

Notably, the present invention is not limited to the combination of the dexterous manipulation device elements as listed above and may be assembly in any combination of the elements as described herein.

Other aspect of the invention as disclosed infra.

### BRIEF DESCRIPTION OF THE DRAWINGS

Aspects of the present disclosure may be better understood with reference to the following drawings. Components of the drawing are not necessarily to scale, emphasis instead being placed upon clearly illustrating the principles of the present disclosure. Moreover, the drawings, like reference numerals designate corresponding parts throughout the several views.

FIG. 1 a perspective view of a surgeon and a patient in a clinical environment;

FIG. 2 illustrates a simulated RVC procedure;

FIGS. 3A-3B illustrate a dexterous manipulation device according to an exemplary embodiment of the present disclosure;

FIG. 4 illustrates a disc element of the dexterous manipulation device of FIGS. 3A-3B;

FIGS. 5A-5B compare aperture orientation of disc elements relative to contact surface (length) of a prior art compared to that of the exemplary embodiment of the present disclosure;

FIGS. 6A-6C a disc element of the dexterous manipulation device according to another exemplary embodiment of the present disclosure;

FIGS. 7A-7C illustrate a dexterous manipulation device having the disc elements of FIGS. 6A-6C;

FIG. 8 illustrates a wire assembly according to the exemplary embodiment of FIGS. 6A-6C;

FIG. 9 illustrates attachments to the dexterous manipulation device according to an exemplary embodiment of the present disclosure;

FIGS. 10A-10B illustrate the drive mechanism according to an exemplary embodiment of the present disclosure;

FIGS. 11A-11B illustrate a comparison between a pulley and screw actuation device;

FIG. 12 illustrates a graph of the difference of push-pull wire displacement;

FIGS. 13A-13B illustrate the drive mechanism relative to the wires of the system according to an exemplary embodiment of the present disclosure;

FIG. 14 illustrates the surgical system according to an exemplary embodiment of the present disclosure;

FIGS. 15A-15B show a model of the system according to an exemplary embodiment of the present disclosure; FIG. 15A shows a scale-up model and FIG. 15B shows a real scale model; and

FIG. 16 shows an exemplary two degree of freedom user interface control according to an exemplary embodiment of the present disclosure.

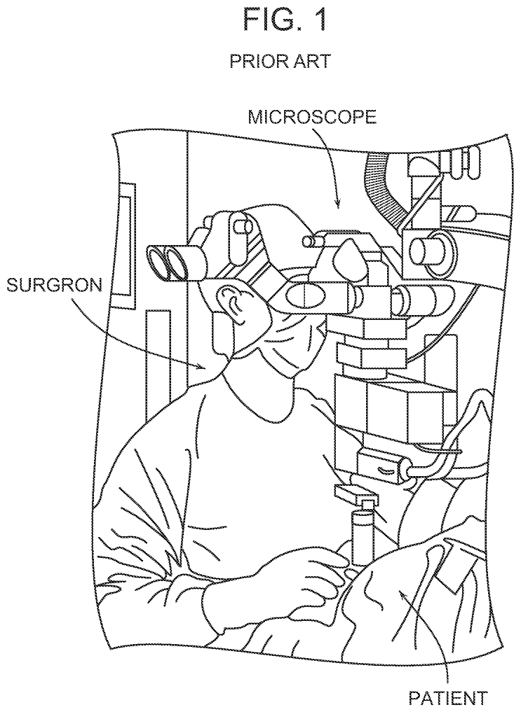

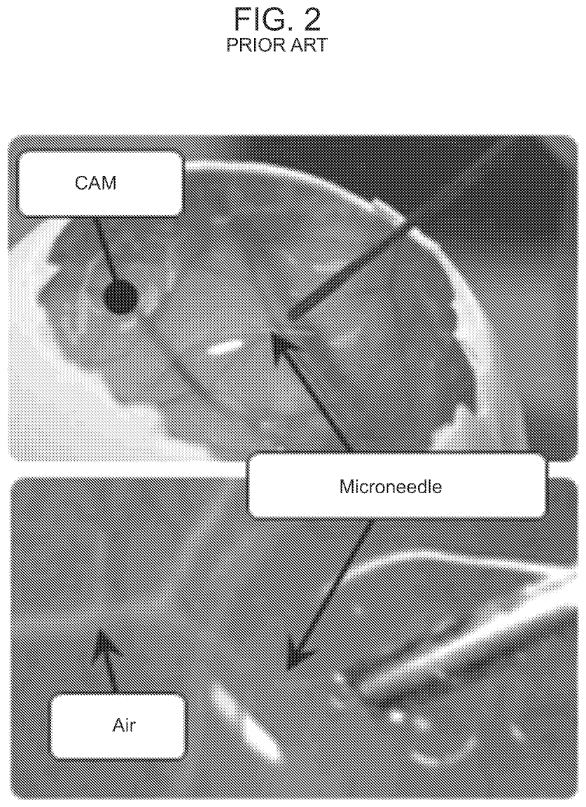

### DETAILED DESCRIPTION

The terminology used herein is for the purpose of describing particular embodiments only and is not intended to be limiting of the invention. As used herein, the singular forms “a”, “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof. As used herein, the term “and/or” includes any and all combinations of one or more of the associated listed items.

Unless specifically stated or obvious from context, as used herein, the term “about” is understood as within a range of normal tolerance in the art, for example within 2 standard deviations of the mean. “About” can be understood as within 10%, 9%, 8%, 7%, 6%, 5%, 4%, 3%, 2%, 1%, 0.5%, 0.1%, 0.05%, or 0.01% of the stated value. Unless otherwise clear from the context, all numerical values provided herein are modified by the term “about.”

Certain exemplary embodiments will now be described to provide an overall understanding of the principles of the structure, function, manufacture, and use of the devices and methods disclosed herein. One or more examples of these embodiments are illustrated in the accompanying drawings. Those skilled in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

It will be appreciated that the terms “proximal” and “distal” may be used throughout the specification with reference to a clinician manipulating one end of an instrument used to treat a patient. The term “proximal” refers to the portion of the instrument closest to the surgeon and the term “distal” refers to the portion located furthest from the surgeon. It will be further appreciated that for conciseness and clarity, spatial terms such as “vertical,” “horizontal,” “up,” and “down” may be used herein with respect to the illustrated embodiments. However, surgical instruments may be used in many orientations and positions, and these terms are not intended to be limiting and absolute.

In the embodiments described herein below, devices and methods are provided for ocular surgeries that allow a user to manipulate a sub-millimeter intra ocular robotic device. That is, the present disclosure provides a snake-like manipulator at a distal end of a surgical instrument to provide flexible access to a retina of an eye. As a result of improving dexterity, the accuracy and efficiency of diagnostic or therapeutic capabilities in ophthalmology may be enhanced, thereby providing tissue access from an optimal surgical angle.

Notably, the devices and methods disclosed herein may be used with a variety of surgical devices, including measuring devices, sensing devices, locator devices and insertion devices, etc. Additionally, the device may be used in a variety of procedures, such as sinus surgery, cochlear implant surgery, subglottic and vocal cord procedures and intra-cardiac procedures. The exemplary embodiments described herein generally relate to a robotic device for performing intraocular surgery.

### Dexterous Manipulation Device First Embodiment

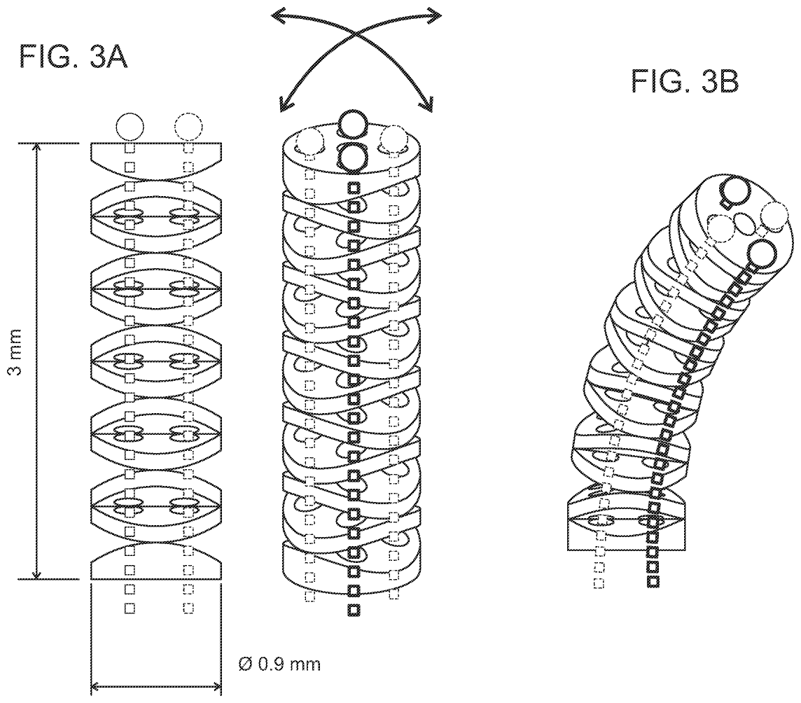

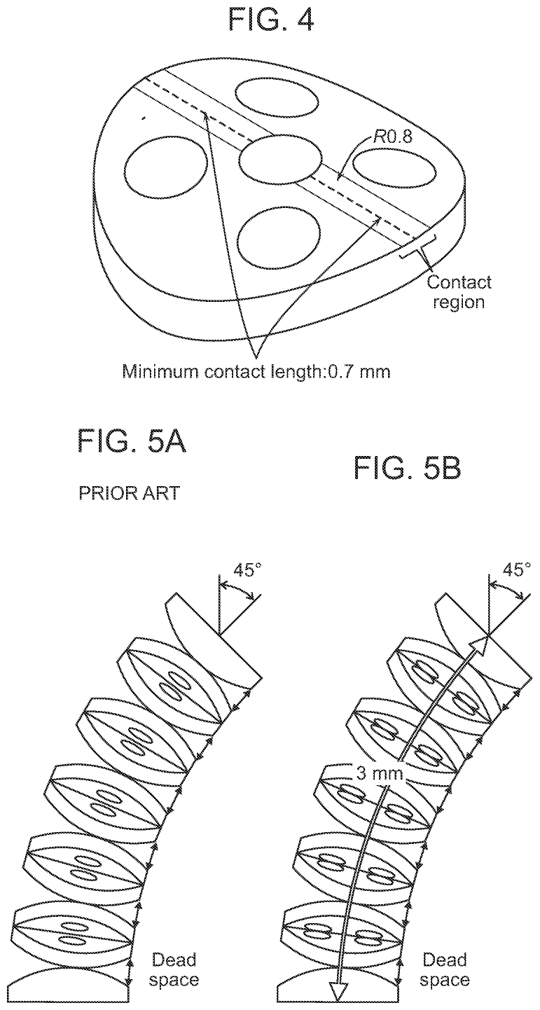

One exemplary embodiment of the present disclosure provides a device for performing intraocular surgery. In particular, as shown in FIGS. 3A-3B and FIG. 4, a dexterous manipulation device of the present disclosure may include a plurality of disc elements. Each of the disc elements has a curved top surface and a corresponding curved bottom surface. The axis of the top surface is orthogonal to that of the bottom surface. Additionally, the device includes actuation wires or cables threaded through apertures formed through each disc element to hold the stacked disc elements together in a snake-like formation by pretension. An optimal pretension level to apply on the device may be determined using Hertz theory. The disc elements may be fabricated from a variety of different materials include metal (e.g. brass or stainless steel) and may be micro-machined.

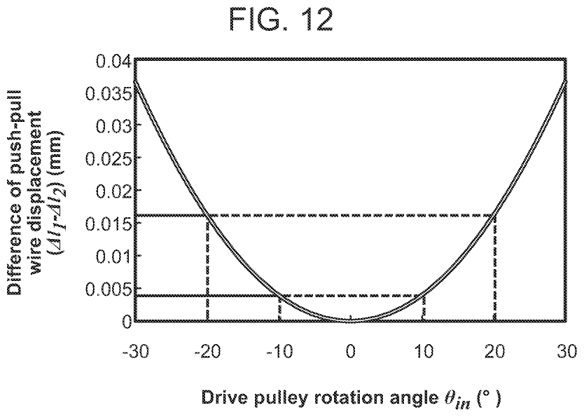

As shown in FIG. 3A, the disc elements are stacked alternating with the curved top and bottom surfaces of adjacent or neighboring disc elements forming a rolling joint. For example, as shown in FIGS. 3A-3B, 12 disc elements may be stacked, but the present disclosure is not limited thereto. The embodiments described herein will reference 12 stacked disc elements merely as an example. The disc elements provide 2 degrees of freedom (DOF) bending joints that are actuated by the actuation wires. For example, four wires may be threaded through the apertures of disc elements. The center aperture may be used to accommodate a micropipette, or to pass a wire for micro forceps actuation. Each of the apertures through which the wires are inserted may have a diameter of about 0.2 mm Nitinol wires may be used with a diameter of about 0.125 mm The overall diameter of the dexterous manipulation device may be less than or equal to about 0.9 mm and the embodiment shown in FIG. 3A provides an overall length of the stacked disc elements as about 3 mm. Each disc element may bend about 7.5 degrees thus providing a total of about 45 bending degree. Due to the curved surfaces of each disc element, in other words, due to the curved top surface and the corresponding curved bottom surface configuration, the disc elements contact each other when stacked. To reduce the contact stress between the neighboring disc elements, the present disclosure increases the contact length of the disc elements.

As shown in FIG. 4, by disposing the center aperture offset from the four wire apertures, the contact region on each disc is increased. For example, the minimum contact length may be increased to about 0.7 mm compared to 0.3 mm of a conventional stack of disc element. That is, conventionally, 3 apertures are aligned through the center of the disc element thus providing a minimum contact length of merely about 0.3 mm FIGS. 5A-5B compare aperture orientation of disc elements relative to contact surface (length) of a prior art compared to that of the exemplary embodiment of the present disclosure. The thickness of the disc elements in this configuration is about 0.25 mm and thus with 12 disc element shown, the extension length of the stacked disc elements is about 3 mm.

### Dexterous Manipulation Device Second Embodiment

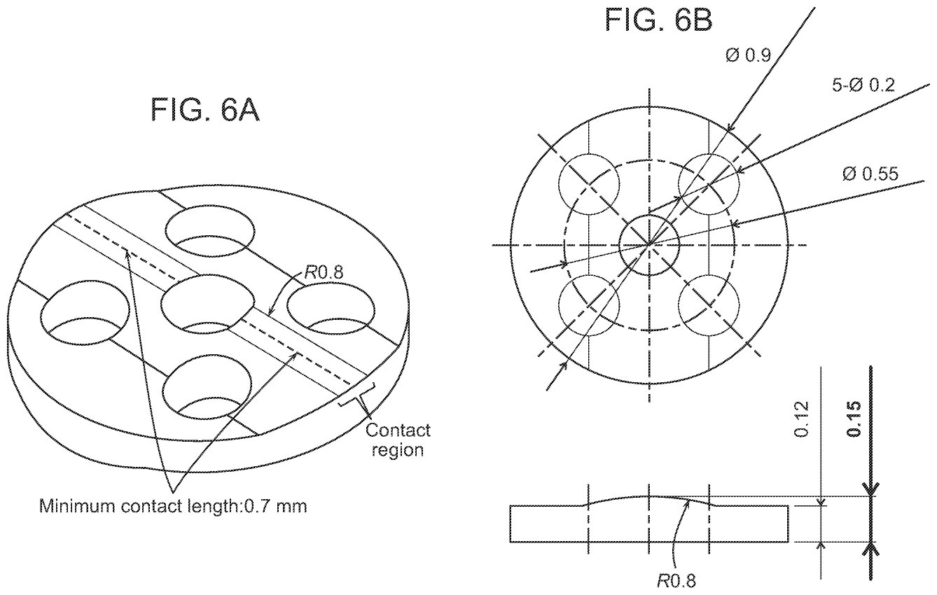

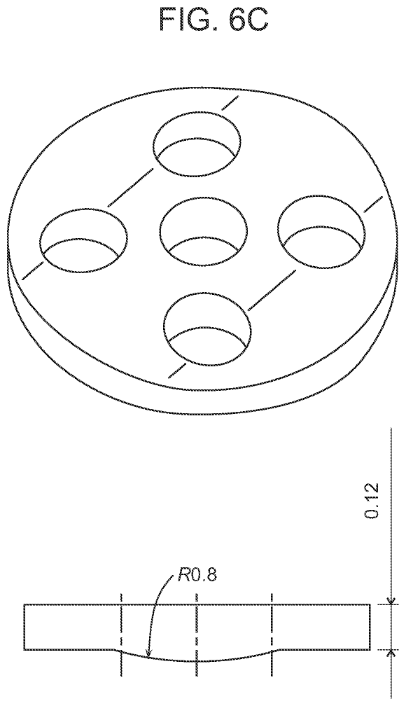

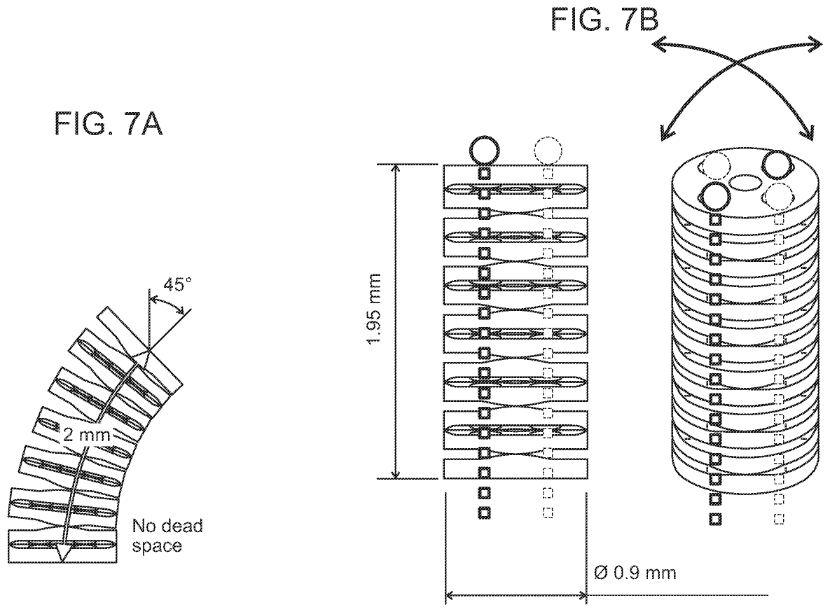

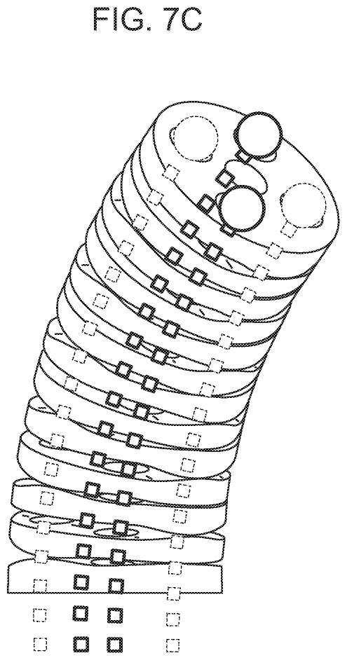

According to another exemplary embodiment of the present disclosure, a more compact dexterous manipulation device is proposed. The compact configuration of the device further reduces the contact stress between disc elements. In particular, as shown in FIGS. 6A-6C, the disc elements may each have a partially curved top surface and a partially curved bottom surface, the curve of which corresponds to the top surface. As shown in FIG. 6B, the outer diameter of the disc element may be about 0.9 mm and an inner diameter through the center of the four apertures may be about 0.55 mm Additionally, the thickness of the curved portion of the disc element may be about 0.15 mm while the non-curved portion of the disc element may be about 0.12 mm By merely curved a portion of the disc surface, the contact region between the neighboring disc elements may be further reduced, as shown in FIGS. 7A-7C. In this configuration, each as shown in FIG. 7A and FIG. 7C, no gap is provided between the pair of disc elements. Additionally, as shown in FIG. 7A, the extension of this configuration is about 2 mm while still providing about a 45 bending degree.

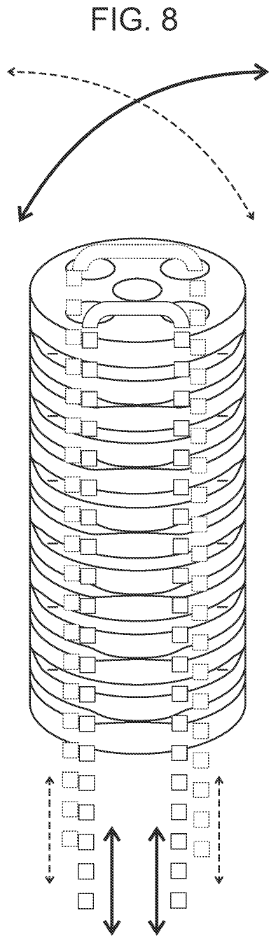

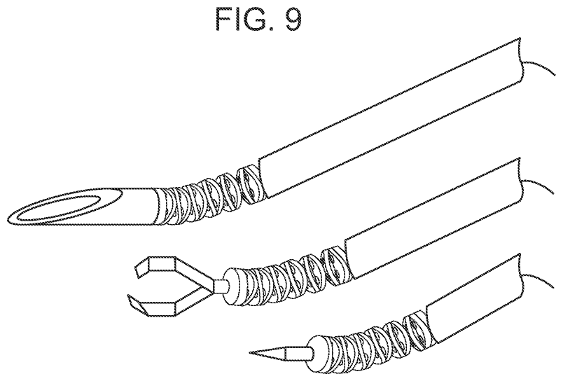

The wire or cable configuration in this embodiment is the same as that of the previous embodiment and thus a detailed description thereof will be omitted. FIG. 8 provides an illustration of the wire assembly for the embodiment of FIGS. 6A-6C. As shown, the wires are thread through the four outer apertures of each disc element to thus slide there through during actuation. This wire assembly method improves ease of assembly and fixes the wires to the disc elements as well as simplifies the tip of an instruments.

Additionally, in both the first and second embodiments described herein above, a distal end of the device may include an instrument tip. For example, as shown in FIG. 9, the distal end may include a needle tip, forceps, a pipette, an intro-ocular device, or a gripper. Alternately, such instruments or drive wire of an end-effector may be guided and protrude out of the center aperture of the stacked disc elements.

### Drive Mechanism

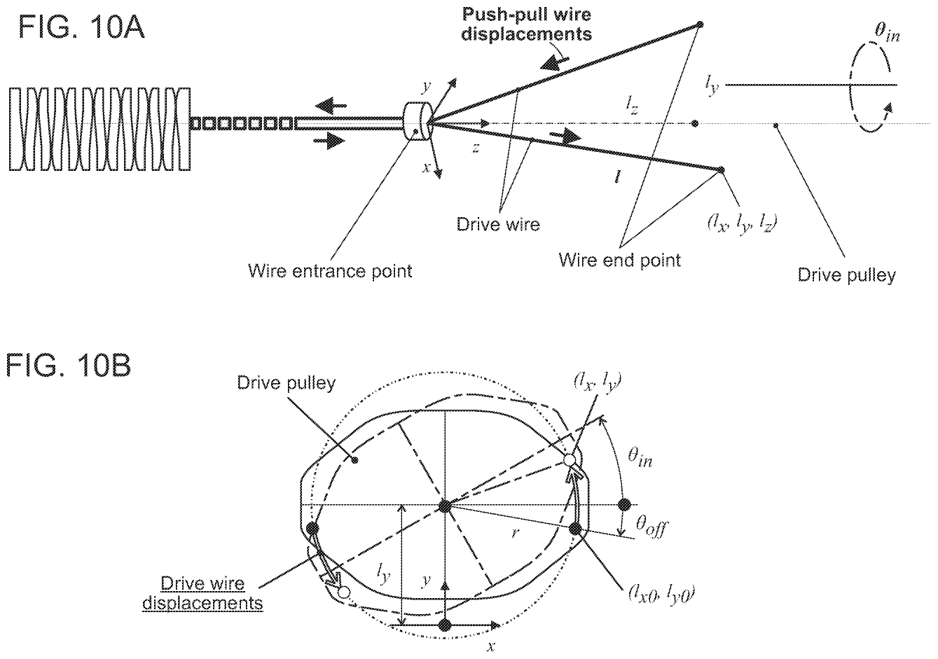

FIGS. 10A-FIG. 14 illustrate the drive mechanism of the surgical system described herein. In particular, the surgical system may include the dexterous manipulation device described above in communication with the drive mechanism. In particular a body unit or instrument shaft may be mated or disposed between the dexterous manipulation device and the drive mechanism. The wires or cables threaded through the disc elements of the dexterous manipulation device are threaded into the drive mechanism housing and actuated by the drive motor therein. This drive mechanism and pulley system will be described in further detail herein below.

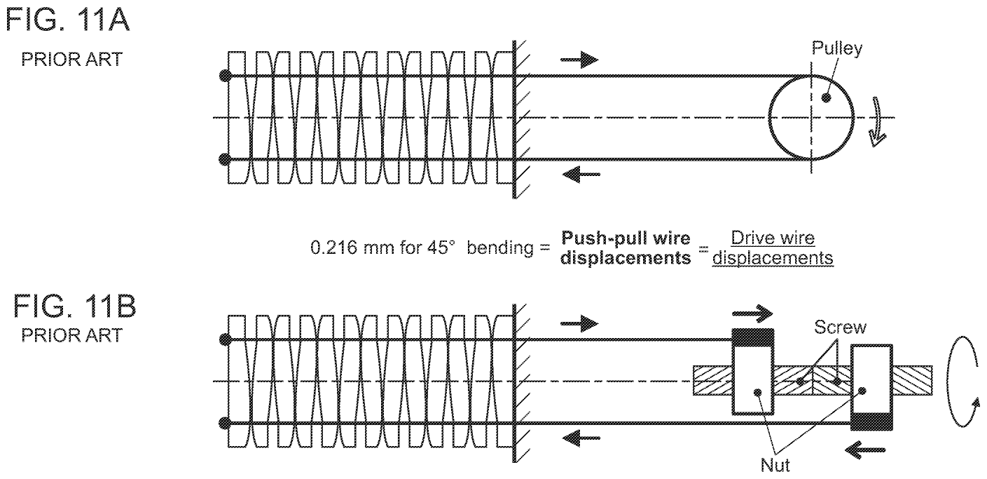

FIG. 10A illustrates a 3D drawing of the wire drive mechanism of the present disclosure and FIG. 10B provides an x-y plane 2D drawing of the wire drive mechanism of the present disclosure. As shown in FIG. 10A, the drive pulley of the drive mechanism is specifically mounted at a right angle relative to the actuation direction, unlike a conventional pulley drive mechanism. That is, FIGS. 11A-11B illustrate a conventional wire drive mechanism. In particular, FIG. 11A illustrates a rotational pulley type of wire drive mechanism and FIG. 11B illustrates a lead screw type wire drive mechanism. The push-pull wire displacement of the conventional mechanisms is only about 0.2 mm for a drive with about 0.9 mm diameter and 45 degree bending motion range. Based on such push-pull wire displacement, it is difficult to maintain the accuracy of bending angle control. Conversely, in the present disclosure, as shown in FIGS. 10A-10B, the wire moves in the x-y plane by the pulley rotation and due to the mounting location of the drive mechanism, the push-pull wire displacement is capable of being increased compared to the convention configurations.

In further detail, the wire length between the wire entrance point into the drive mechanism and the wire end point changes by the pulley rotation. The relationship between the drive pulley rotation angle θᵢₙ and the wire length l is obtained using the following equation:

$$\begin{matrix}l = \sqrt{{l}_{x}^{2} + {l}_{y}^{2} + {l}_{z}^{2}} \\ = \sqrt{{( r cos ( {θ}_{in} - {θ}_{off} ) )}^{2} + {( {l}_{Y} + r sin ( {θ}_{in} - {θ}_{off} ) )}^{2} + {l}_{Z}^{2}}\end{matrix} \qquad ( 1 )$$

wherein r is the drive pulley radius, θ_{off} is the offset angle of the wire end point on the pulley, l_{y} is the y-direction distance of the pulley center, and l_{z} is the z-direction distance from the origin to the end point of the wire on the pulley.

The below Table 1 shows the motion range and displacement of the wire drive mechanism of the present disclosure.

TABLE 1

| | |
|---|---|
| | Motion ranges and |
| Items | Displacements |

| | | |
|---|---|---|
| Drive pulley rotation angle θᵢₙₚᵤₜ | ±10° | ±20° |
| Drive wire displacement rθᵢₙₚᵤₜ | ±0.87 mm | ±1.75 mm |
| Push-pull wire displacement Δlᵢ | ±0.22 mm | ±0.44 mm |
| Dexterous tip bending angle θₒᵤₜₚᵤₜ | ±45° | ±90° |

As shown in Table 1, the drive wire displacement is about four times greater than the push-pull wire displacement. As shown in FIG. 12, the difference between two push-pull wire displacements is under 20 μm at the drive pulley rotation angle of about 20 degrees. Accordingly, the system of the present disclosure is capable of enabling two-motor actuation with two degrees of freedom.

Furthermore, the wire assembly also maintains the disc elements stacked together based on a pretension of the wire. In other words, the disc elements are held together based on such a pretension. The relationship between the input torque T and the wire F may be determined using the following equation:

$$T = {F}_{r} r = F \frac{{l}_{xy}}{l} r sin {θ}_{A} \\ where {F}_{r} = {F}_{xy} sin {θ}_{A} , {F}_{xy} = F \frac{{l}_{xy}}{l} , cos {θ}_{A} = \frac{{l}_{xy}^{2} + {r}^{2} - {l}_{Y}}{2 {l}_{xy} r} . \qquad ( 2 )$$

$$When T = FR , R is expressed as follows : \\ R = \frac{{l}_{xy}}{l} r sin {θ}_{A} \qquad ( 3 )$$

wherein R is a virtual radius.

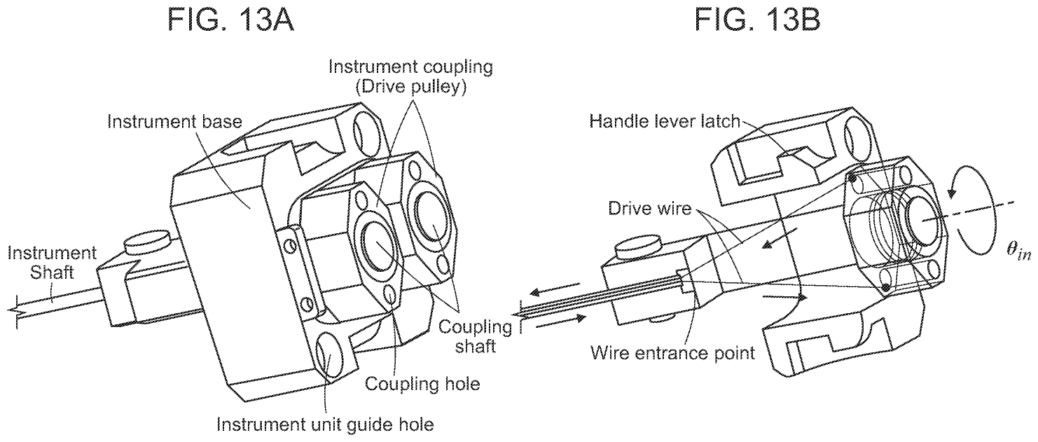

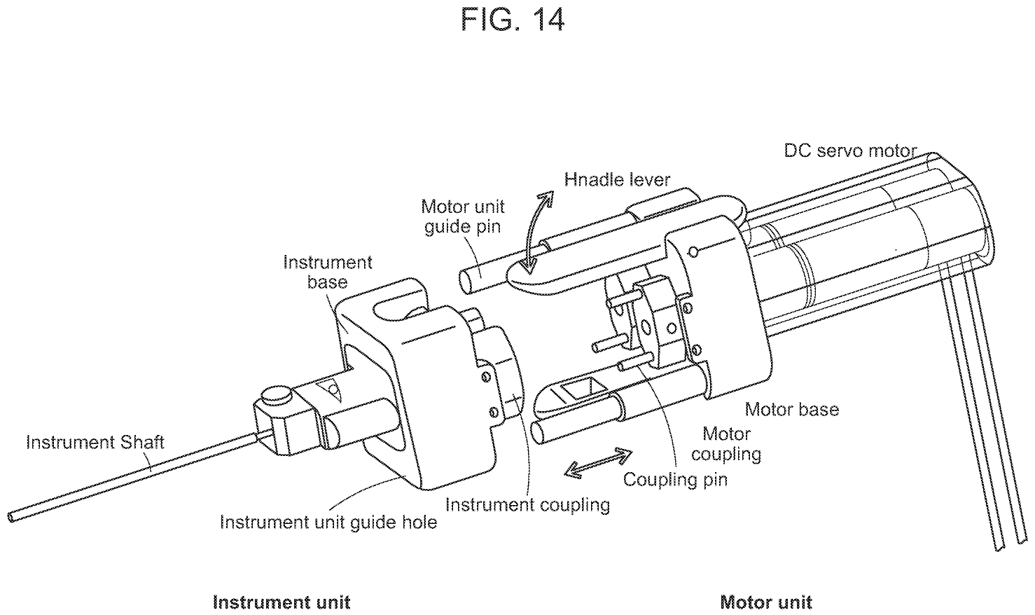

Moreover, FIGS. 13A-13B and FIG. 14 illustrate the instrument and motor unit design. In particular, as shown in FIG. 13A, the drive pulley is specifically disposed at a right angle relative to the actuation direction. The instrument shaft may be considered as the body unit that is disposed between the dexterous manipulation unit and the drive mechanism. Additionally, FIG. 13B illustrates how the wires enter the housing and then separate to each end of the drive pulley to be actuated as the drive pulley is rotated. In other words, as shown in FIG. 13B, the rotation of the drive pulley causes the movement of the wires in the directions shown by the arrows.

FIG. 14 illustrates how the instrument base and dexterous manipulation device are detachable from the motor unit. This detachable design advantageously allows for ease of cleaning, sterilization, and attachment of different surgical tools. For attachment of the separate units, a motor guide pin is provided on the motor base that slides into a groove of the instrument base. Additionally, a motor couple pin is guided into the instrument unit guide hole to thus couple the motor base with the instrument base. The coupling direction is shown by the arrows in FIG. 14. The handle lever of the motor unit is lifted upwards (shown by the arrow) to decouple the units from each other when desired. In other words, the handle lever released the motor unit guide pins from the grooves of the instrument base to thus allow separation of the units. As also shown, the instrument shaft extends out from the instrument base and the dexterous manipulation device is formed at an end thereof.

### Experiments

For evaluation of the device described herein, a 5:1 scale-up model of the device was built using rapid prototyping together with actual-size models of the instrument and motor units. The experiments provide evidence of the bending motions of the dexterous manipulation devices described herein and the functions of the drive mechanism.

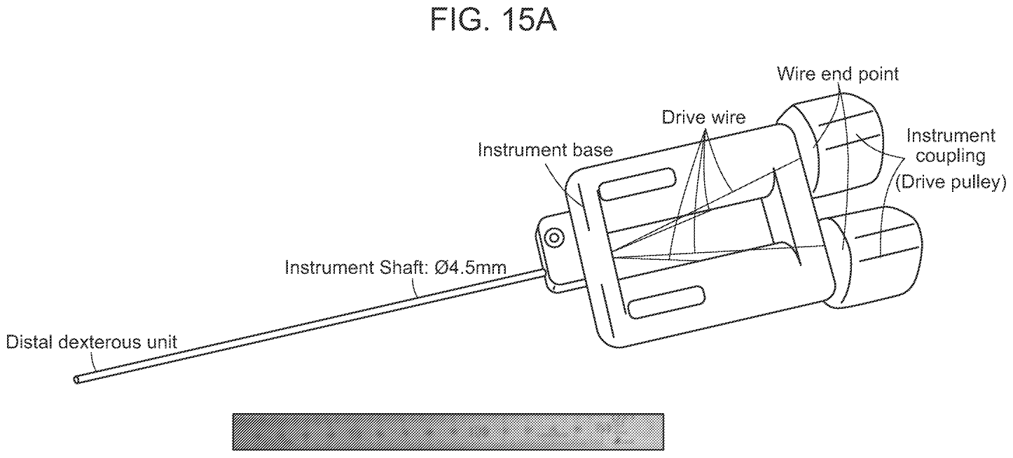

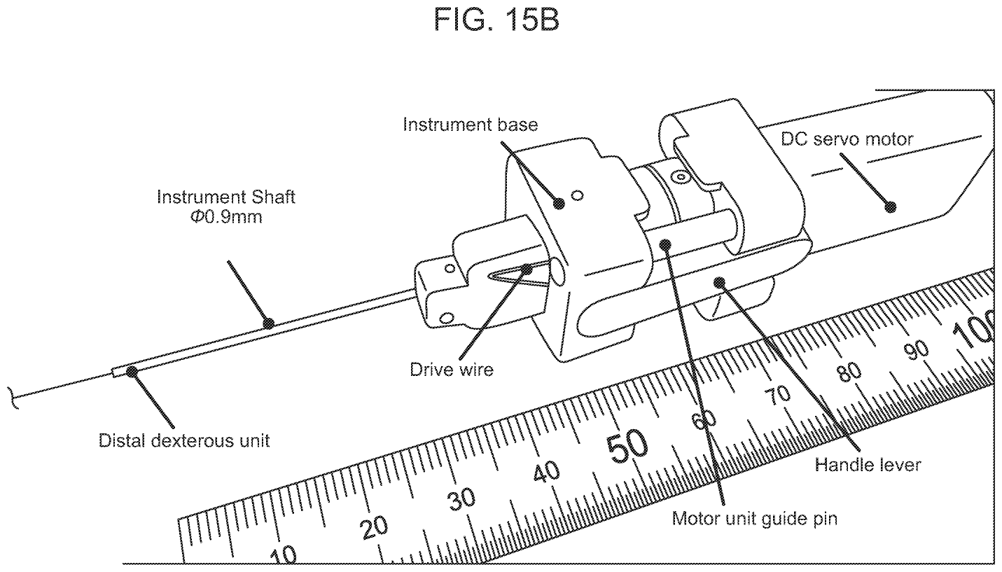

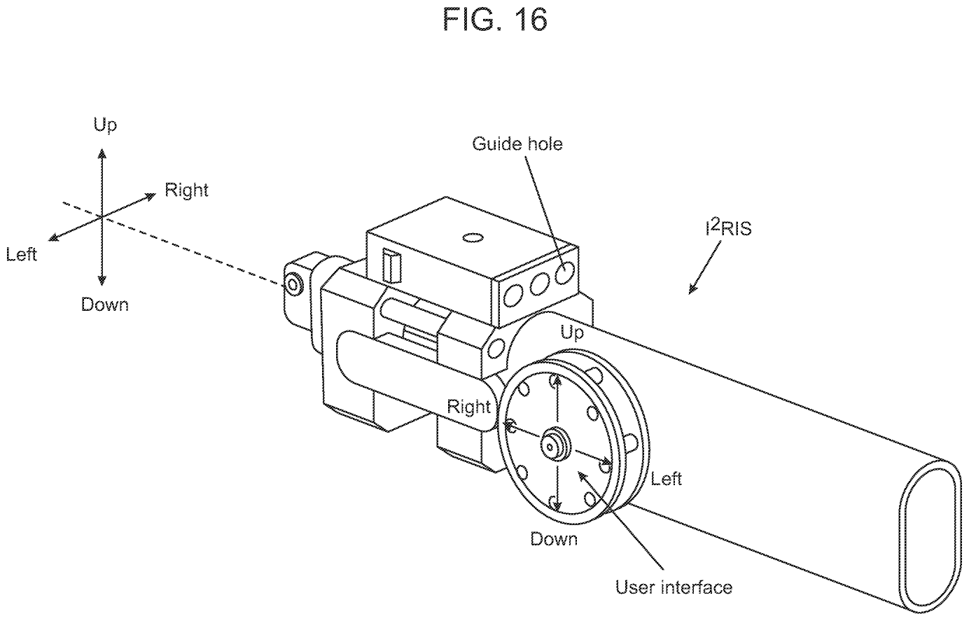

First, for the scale-up model of the dexterous manipulation device, the drive wires were about 0.45 mm in diameter and the apertures through the disc elements were about 1 mm to 0.6 mm to maintain the ratio of the wire to hole diameter. The 45 degree yaw and pitch bending motions were performed by rotation of the drive pulley (shown in FIGS. 15A-15B). The maximum motion range was determined to be about 90 degrees. The range of motion may be increased by increasing the number of stacked disc elements.

FIG. 15B shows the full system with actual-size models of the instrument and motor units and FIG. 15A shows the scale-up model described above. In particular, in the models the drive wires were about 0.15 mm in diameter. The distal ends of the wires were fixed by knots and the proximal ends were fixed at the drive pulley. The restitution force by the wire tension allowed for the instrument couplings to be returned to original positions automatically thus easing the alignment with the motor couplings when coupling the instrument unit with the motor unit. With this setup, the bending angle of the dexterous manipulation device was tested with the operation of the drive pulley. The results shows that a motion range of about 45 degrees was obtained by a command angle of the drive pulley of about 30 degrees or less when a payload was about 34 mN thus showing the effectiveness of the configuration of the system described herein.

### User Control Interface

According to another aspect of the present disclosure, a user interface may be additionally provided to the surgical system to control the two degrees of freedom movement. FIG. 16 shows an example of such a user interface. For example, a joystick, tactile switch, trackball, mouse, force sensors, tactile sensors, or the like as well as a combination thereof may be used as interfaces of the system to provide control of two degrees of motion. Thus, the bending function of the system may be controlled by the tactile user interface and may be integrated with a steady hand eye robot (SHER). This integration allows for execution of 3D targeting tasks within the confined intraocular space of the eye. Notably, the dexterous manipulation device may also be mechanically detachable from the SHER to change various surgical tools.

The integration of the SHER with the system described herein allows a surgeon or operate to control the five degrees of freedom tool tip position single-handedly. That is, a three degree of motion may be performed by holding the dexterous manipulation device in combination with the drive mechanism (attached to the SHER) and the two degree of freedom bending motion may be performed by orienting the tip of the dexterous manipulation device using the tactile user interface.

The system described herein provides a more compact instrument that is capable of approaching a surgical target from suitable directions and operate delicate tissues. The reduced size of the dexterous manipulation device reducing contact stress between neighboring disc elements. The compact design allows the device to also be integrated into a cooperatively-controller steady hand eye robot unit and provides high dexterity for micromanipulations inside the eye during surgery. The specific disposition of the apertures formed through the disc elements also aids in reducing the contact stress between neighboring discs. Additionally, by mounting the drive pulley of the drive mechanism at a right angle relative to an actuation direction, the system is capable of achieving higher accuracy in manipulation control. The dexterous manipulative device is also detachable from the drive mechanism thus facilitating easier cleaning, sterilization, and attachment of surgical tools.

The many features and advantages of the disclosure are apparent from the detailed specification, and thus, it is intended by the appended claims to cover all such features and advantages of the disclosure which fall within the true spirit and scope of the disclosure. Further, since numerous modifications and variations will readily occur to those skilled in the art, it is not desired to limit the disclosure to the exact construction and operation illustrated and described, and accordingly, all suitable modifications and equivalents may be resorted to, falling within the scope of the disclosure.

1. A dexterous manipulation device, comprising:
a plurality of disc elements, each having a curved top surface and a corresponding curved bottom surface; and
actuation wires threaded through apertures of each disc element,
wherein the disc elements are stacked alternating with the curved top and bottom surfaces of adjacent disc elements forming a rolling joint, and
wherein the dexterous manipulation device has a total of 45 degrees of bending motion with two degrees of freedom.

2. The dexterous manipulation device of claim 1, wherein outer apertures of each disc element are disposed outside of a contact region between neighboring disc elements.

3. The dexterous manipulation device of claim 2, wherein a center apertures of each disc element is disposed inside the contact region between neighboring disc elements.

4. The dexterous manipulation device of claim 1, wherein each disc element is about 0.2 mm thick.

5. The dexterous manipulation device of claim 1, wherein the apertures formed through each disc element are about 0.2 mm in diameter.

6. The dexterous manipulation device of claim 1, wherein the device is robotically controlled.

7. The dexterous manipulation device of a claim 1, wherein the device is less than about 0.9 mm in diameter.

8. The dexterous manipulation device of claim 1, wherein the length of the stacked disc elements is about 3 mm or less.

9. The dexterous manipulation device of claim 1, wherein a distal end of the device includes one of a needle tip, forceps, a pipette, an intra-ocular device, or a gripper.

10. The dexterous manipulation device of claim 1, wherein disc elements provided at a proximate end of the device include about 9 apertures to provide four degrees of freedom.

11. A surgical system, comprising:
a dexterous manipulation device including at least one bending portion actuated by wires; and
a drive mechanism mounted at a right angle relative to an actuation direction of the dexterous manipulation device.

12. The system of claim 11, wherein the bending portion is a plurality of disc elements each having a curved top surface and a corresponding curved bottom surface and wherein the wires are threaded through apertures of each disc element

13. The system of claim 11, wherein the drive mechanism is detachable from the dexterous manipulation device.

14. The system of claim 11, further comprising a body unit mated between the drive mechanism and the dexterous manipulation device.

15. The system of claim 13, wherein the drive mechanism includes a housing, a motor within the housing, and a plurality of pulleys.

16. A dexterous manipulation device, comprising:
a plurality of disc elements, each having a partially curved top surface and a partially curved bottom surface corresponding to the curved top surface; and
actuation wires threaded through apertures of each disc element,
wherein the disc elements are stacked alternating with the curved top and bottom surfaces of adjacent disc elements forming a rolling joint,
wherein the dexterous manipulation device has a total of 45 degrees of bending motion with two degrees of freedom.

17. The dexterous manipulation device of claim 16, wherein neighboring disc elements maintain constant contact with each other.

18. The dexterous manipulation device of claim 17, wherein the apertures formed through each disc element are arranged to provide a minimum contact length of about 0.7 mm between neighboring disc elements.

19. The dexterous manipulation device of claim 17, wherein the length of the stacked disc elements is about 2 mm or less.

20. The dexterous manipulation device of claim 16, wherein each disc element is about 0.2 mm thick.

21-22. (canceled)
